(19) United States
(12) Patent Application Publication
(10) Pub. No.: US 2006/0160161 A1
(43) Pub. Date: Jul. 20, 2006

(54) Methods for assessing antibodies to neurodegenerative disease-associated antigens

(75) Inventors: Danka Pavliakova, Montebello, NY (US); Eric Hryhorenko, Hilton, NY (US); Stephen Hildreth, Clarks Summit, PA (US)

Correspondence Address: LAHIVE & COCKFIELD, 28 STATE STREET, BOSTON, MA, 02109, US

(73) Assignee: ELAN PHARMACEUTICALS, INC., South San Francisco, CA (US)

(21) Appl. No.: 11/260,047

(22) Filed: Oct. 26, 2005

Related U.S. Application Data
(60) Provisional application No. 60/622,525, filed on Oct. 26, 2004.

(51) Int. Cl. G01N 33/537; G01N 33/53; G01N 33/543

(52) U.S. Cl. 435/7.92

(57) ABSTRACT

The invention provides methods for assessing immunoglobulins directed to neurodegenerative disease-associated antigens. Methods of the instant invention involve specific, sensitive and precise identification of Ig classes of antibodies present in a test sample that are capable of binding an immobilized neurodegenerative disease-associated antigen. The enhanced assessment of antibodies directed to specific neurodegenerative disease-associated antigens that is enabled by the invention improves both monitoring of neurodegenerative disease treatment and prediction of neurodegenerative disease progression.

### RELATED APPLICATION

This application claims priority to U.S. patent application Ser. No. 60/622,525, filed on Oct. 26, 2004, the entire content of which is hereby incorporated by reference.

### BACKGROUND OF THE INVENTION

Alzheimer's disease (“AD”) is a neurodegenerative disorder characterized by the occurrence of amyloid plaques, neurofibrillary tangles and significant neuronal loss. Amyloid protein, the main component of senile plaques, has been implicated in the pathogenesis of Alzheimer's disease (Selkoe (1989) Cell 58:611-612; Hardy (1997) Trends Neurosci 20:154-159). P-Amyloid has been shown to be both directly toxic to cultured neurons (Lorenzo and Yankner (1996) Ann. NY Acad. Sci. 777:89-95) and indirectly toxic through various mediators (Koh, et al. (1990) Brain Research 533:315-320; Mattson, et al. (1992) J. of Neurosciences 12: 376-389). Additionally, in vivo models, including the PDAPP mouse and a rat model have linked β-amyloid to learning deficits, altered cognitive function, and inhibition of long-term hippocampal potentiation (Chen, et al. (2000) Nature 408, 975-985; Walsh, et al. (2002) Nature 416:535-539). Therefore, a great deal of interest has focused on therapies that alter the levels of β-amyloid to potentially reduce the severity or even abrogate the disease itself.

One AD treatment strategy that has recently emerged in response to successful studies in PDAPP mouse and rat experimental models, is that of immunization of individuals to either provide immunoglobulins (as in the case of passive immunization, wherein immunoglobulins generated outside of a subject are directly administered to a subject) or to generate immunoglobulins (active immunization, wherein the immune system of a subject is activated to produce immunoglobulins to an administered antigen) specific to β-amyloid. These antibodies would in turn help reduce the plaque burden by preventing β-amyloid aggregation (Solomon, et al. 1997 Neurobiology 94:4109-4112) or stimulating microglial cells to phagocytose and remove plaques (Bard, et al. (2000) Nature Medicine 6: 916-919). The utility of active immunization was initially shown by Schenk, et al. in 1999 (Schenk, et al. (1999) Nature 400:173-177) using a synthetic 42 amino acid version of β-amyloid (Aβ₄₂, in later studies called AN1792). PDAPP mice receiving Aβ₄₂ immunizations either had reduced or no plaque formation depending on the age of the animal. Since that study, numerous animal studies have been performed examining the effectiveness of Aβ₄₂ immunization (Das, et al. (2001) Neurobiology of Aging 22, 721-727; Sigurdsson, et al. (2001) American J. of Pathology. 159: 439-447; Monsonego, et al. (2001) PNAS 98: 10273-10278).

Studies involving treatment of Alzheimer's patients via immunization with the 42 amino acid peptide have rapidly advanced to clinical trials. Phase I clinical safety studies (single and multiple dose) using AN1792 to immunize humans have been completed. The results demonstrated the formulation (AN1792 plus QS-21 adjuvent) was well-tolerated in humans. However, a phase II clinical trial using AN1792 to immunize humans with mild to moderate Alzheimer's was terminated early due to symptoms in a small subset of patients indicative of meningoencephalitis (Schenk (2002) Nature 3:824-828). Investigations are currently underway to determine the causes of encephalitis. Understanding the results from this clinical study is important for the continuated development and administration of Aβ₄₂ immunotherapies. Defining the specific nature of immunological responses to Aβ₄₂ immunization can help to assure the safety of future active immunization programs, in addition to providing information that can be used to improve immunotherapeutic formulations and treatment protocols.

### SUMMARY OF THE INVENTION

The present invention addresses the documented need for improved methods for assessing immunoglobulins directed to neurodegenerative disease-associated antigens (NDAAs). Methods of the instant invention involve specific, sensitive and precise identification of Ig classes or subclasses of antibodies present in a test sample that are capable of binding an immobilized NDAA. The enhanced assessment in a subject of antibodies directed to specific NDAAs provided by the invention advances both monitoring of neurodegenerative disease treatment and prediction of neurodegenerative disease progression. While monitoring of Alzheimer's disease (AD) treatment and progression is featured in many embodiments, the methods of the instant invention can be applied to the full range of known neurodegenerative disorders.

In exemplary embodiments, the methods and kits of the invention feature the use of a particular neurodegenerative disease-associated proteins (or protein fragment) as an assay reagent, the antigen being the same as that to which individuals have been exposed for the purpose of triggering an immune response (“active immunization”). In alternative embodiments, the methods and kits of the invention may be used to identify the antibodies directed to NDAAs present in individuals who have been treated with such antibodies derived from an exogenous source (“passive immunization”). Antibody production to NDAAs such as β-amyloid will tend to vary across individuals for any number of stochastic, genetic and environmental reasons, resulting in a wide range of possible immune responses to NDAA treatment ranging from no detectable immune response to full production of IgM, IgA, IgG (IgG₁, IgG₂, IgG₃, and IgG₄) and IgE anti-NDAA antibodies. For AD, the repertoire of anti-Aβ antibodies present in an individual has been observed to correlate with the outcome of certain AD therapies (e.g. immunotherapies). The ability of the present invention to discern with precision, sensitivity and rapidity the full range of Ig classes and subclasses of such antibodies in the bodily fluids of an individual constitutes a decided advance over previously existing technologies.

Accordingly, the invention has several advantages which include, but are not limited to, the following:

detection methods of improved fidelity for rapidly determining the Ig classes and subclasses of anti-NDAA antibodies present in the bodily fluids of an individual.

kits that allow rapid discernment of the Ig classes and subclasses of anti-NDAA antibodies present in the bodily fluids of an individual.

The detection methods and kits described herein offer high levels of specificity and sensitivity for detection of anti-β-amyloid antibodies. Moreover, the invention achieves surprisingly high levels of precision, accuracy and reproducibilty and sensitivity for detection of anti-β-amyloid antibodies, at least in part, to the choice of immobilized antigen, i.e., AN1792. Moreover, quantitation of anti-β-amyloid antibody levels is made more precise than previous methods of quantitating anti-β-amyloid antibody levels through use of internal controls having a predetermined level of detectability or activity in the featured assays. In exemplary embodiments, human sera (or pools thereof) containing predetermined levels of anti-β-amyloid antibodies are used as internal controls in the featured assays. In other exemplary embodiments, mammalian sera (e.g., primate sera) (or pools thereof) containing predetermined levels of anti-β-amyloid antibodies are used as internal controls in the featured assays. Human or mammalian (e.g., primate) sera or sera pools can be obtained from such subject having been immunized with β-amyloid (e.g., Aβ₄₂). In certain embodiments, Cynomoglus monkey anti-β-amyloid antibody-containing sera is used as an internal control in featured assays. Such controls are used to standardize both inter-sample comparison of titer levels and to more accurately define absolute titer levels. Both within day and between day precision have also been assessed in the experiments described herein, resulting in enhanced precision of titer value observation.

In one aspect, the invention provides a method for determining the level of an anti-Aβ antibody of a particular immunoglobulin (Ig) class or subclass produced in a patient immunized with an Aβ immunogenic composition, comprising: contacting a biological sample from the patient with an immobilized Aβ antigen under conditions sufficient for binding of the anti-Aβ antibody to the antigen in the sample, if present, followed by washing to remove unbound biological sample; then contacting the bound anti-Aβ antibody, if present, with an agent capable of specifically binding to the antibody, followed by washing to remove unbound agent; and then quantifying the bound agent, wherein the quantity of agent bound indicates an amount of anti-Aβ antibody present in the biological sample, such that the level of the anti-Aβ antibody of a particular Ig class or subclass is determined. In one embodiment, the Aβ antigen is Aβ1-42. In certain embodiments, the Aβ antigen is AN1792.

In an additional embodiment, the level of the anti-Aβ antibody of a particular Ig class is determined. In another embodiment, the agent capable of specifically binding to the antibody is a second antibody which is detectable. In an additional embodiment, the second antibody is enzyme-linked. In certain embodiments, quantifying the enzyme-linked second antibody comprises detecting an activity of the enzyme-linked antibody, wherein a level of activity indicates a level of bound anti-Aβ antibodies.

In another embodiment, the level of the anti-Aβ antibody of a particular Ig subclass is determined. In an additional embodiment, the agent capable of specifically binding to the antibody is a second antibody. In another embodiment, quantifying the second antibody comprises exposing the second antibody to a third antibody which is detectable. In an additional embodiment, the third antibody is enzyme-linked. In certain embodiments, quantifying the enzyme-linked third antibody comprises detecting an activity of this antibody, wherein a level of activity indicates a level of bound anti-Aβ antibodies.

In an additional embodiment, the anti-Aβ antibody detected by the methods of the invention is of the IgG, IgA, IgM or IgE class. In another embodiment, the anti-Aβ antibody detected by the methods of the invention is of the IgG₁, IgG₂, IgG₃, IgG₄ or subclass.

Another aspect of the invention features a method for determining the efficacy of a β-amyloid immunotherapy in a patient, comprising: assaying for class-specific or subclass specific anti-β-amyloid antibodies in a biological sample from the patient, wherein a level of such isotype-specific or subclass-specific antibodies is determinative of the efficacy of the β-amyloid immunotherapy. In one embodiment, assaying for isotype-specific or subclass specific anti-β-amyloid antibodies comprises contacting the biological sample with immobilized β-amyloid under conditions sufficient for binding of immobilized β-amyloid to class-specific or subclass specific anti-β-amyloid antibodies, if present, followed by washing to remove unbound biological sample; then contacting the bound anti-β-amyloid antibodies, if present, with an enzyme-linked agent which specifically binds to the antibodies, followed by washing to remove unbound agent; and then quantifying the enzyme-linked agent, wherein the quantity of this enzyme-linked agent indicates a level of bound anti-β-amyloid antibodies, such that isotype-specific or subclass specific anti-β-amyloid antibodies are assayed. In some embodiments, the immobilized β-amyloid is Aβ₁₋₄₂. In certain embodiments, the β-amyloid immunotherapy comprises administering a β-amyloid immunogenic composition to said patient. In an additional embodiment, the β-amyloid immunogenic composition comprises Aβ₁₋₄₂ In another embodiment, the β-amyloid immunogenic composition comprises AN1792. In one embodiment, the the immunogenic composition further comprises an adjuvant. In certain embodiments, the adjuvant is STIMULON™ QS-21.

In another embodiment, class-specific antibodies are assayed in the biological sample. In an additional embodiment, IgG, IgA or IgM antibodies are assayed in the biological sample. In certain embodiments, an increase in the level of IgG, IgA or IgM antibodies following initiation of an immunotherapy is determinative of the efficacy of a β-amyloid immunotherapy.

In another embodiment, the level of assayed forms of antibodies present in a biological sample is compared to the level of said antibodies present in a suitable control. In an additional embodiment, subclass-specific antibodies are assayed in the biological sample. In certain embodiments, IgG₁, IgG₂, IgG₃ or IgG₄ antibodies are assayed. In additional embodiments, an increase in the level of IgG₁, IgG₂, IgG₃ or IgG₄ antibodies following initiation of an immunotherapy is determinative of the efficacy of said β-amyloid immunotherapy.

In another embodiment, the biological sample is a serum sample. In certain embodiments, the biological sample is a cerebrospinal fluid (CSF) sample.

In an additional embodiment, the subject has or is at risk for an amyloidogenic disease. In certain embodiments, the subject has or is at risk for Alzheimer's Disease.

Another aspect of the invention features a kit comprising an immobilized Aβ antigen or Aβ antigen suitable for immobilization, an agent capable of specifically binding to an anti-Aβ antibody, and directions for use. In one embodiment, the Aβ antigen is Aβ1-42. In an additional embodiment, the Aβ antigen is AN1792. In another embodiment, the agent is capable of specifically binding to an anti-Aβ antibody of a particular Ig class. In certain embodiments, the agent capable of specifically binding to the anti-Aβ antibody is a second antibody which is detectable. In an additional embodiment, the second antibody is enzyme-linked. In certain embodiments, the kit further comprises agents suitable for detecting activity of the enzyme.

In an additional embodiment, the agent of the kit is capable of specifically binding to an anti-Aβ antibody of a particular Ig subclass. In some embodiments, the agent capable of specifically binding to the anti-Aβ antibody is a second antibody. In another embodiment, the kit further comprises a third antibody capable of binding the second antibody. In an additional embodiment, the third antibody is enzyme-linked. In another embodiment, the kit further comprises agents suitable for detecting activity of the enzyme. In certain embodiments, the anti-Aβ antibody of the kit is of the IgG, IgA, IgM or IgE class. In an additional embodiment, the anti-Aβ antibody of the kit is of the IgG₁, IgG₂, IgG₃, IgG₄ or subclass.

Other features and advantages of the invention will be apparent from the following detailed description and claims.

### DETAILED DESCRIPTION OF THE INVENTION

Specific, sensitive and precise methods have been developed to assess the repertoire of anti-NDAA antibodies that are present in an individual. In certain embodiments of the methods of the invention, specificity, sensitivity and precision are significant due, at least in part, to use of the synthetic Aβ antigen, AN1792. Additionally, the use of anti-β-amyloid antibody-containing sera having predetermined anti-β-amyloid antibody levels (e.g., primate or human anti-β-amyloid antibody-containing sera) as internal standards in certain assay formats allows for normalization of antibody titer between assays, and enhances the overall accuracy of titer determination for test samples assayed by the methods of the instant invention. The rapid performance and scope of Ig classes and/or subclasses assessed by the methods and kits of the instant invention also represent an advance over extant techniques. Use of the methods of the instant invention to determine the Ig classes and/or subclasses of anti-NDAA antibodies present in an individual (e.g., an AD patient), is particularly well suited for monitoring the immunotherapeutic treatment of neurodegenerative disease (e.g., AD) in said individuals. Utilizing the featured assays, the development of anti-NDAA antibodies in a subject immunized against the neurodegenerative disease (“active immunization”, e.g. β-amyloid immunization of AD patients) may be monitored, or the continued presence of anti-NDAA antibodies in an individual treated with such antibodies derived from an exogenous source (“passive immunization”) may be examined. The methods may thus be used to track the progression of Aβ therapy (e.g., immunotherapy) with improved precision.

According to the invention, a biological sample is exposed to an immobilized antigen. The antigen should be capable of binding with an antibody associated with neurodegenerative disease. In certain embodiments, the antigen is β-amyloid (e.g., the antigen may be derived from β-amyloid plaque). In a particular embodiment, the antigen is a synthetic version of the 42 amino acid β-amyloid plaque material (e.g., AN1792).

In one embodiment, the invention provides a method for identifying the Ig class of an antibody associated with AD present in a biological fluid of a subject, comprising the steps of exposing a biological fluid from the subject (whose biological fluid is believed to comprise an antibody that recognizes an AD-associated antigen and belongs to an Ig class) with the immobilized antigen associated with AD, exposing the biological fluid to a detectable binding agent that is capable of specifically binding antibodies belonging to the Ig class, and detecting the agent for the purpose of identifying the antibody as belonging to the Ig class.

In another embodiment, the invention provides a method for monitoring a subject treated with an AD immunotherapy, comprising the steps of exposing a biological fluid from the subject (whose biological fluid is believed to comprise an antibody that recognizes an AD-associated antigen and belongs to an Ig class) with the immobilized antigen associated with AD, exposing the biological fluid to a detectable binding agent that is capable of specifically binding antibodies belonging to the Ig class, and detecting the agent for the purpose of correlating detection of the agent with the presence or development of the antibody of the biological fluid against AD. In certain embodiments, the antibody of the biological fluid being detected is selected from the group consisting of IgG, IgM, IgA and IgE. The methods are particularly suited for detecting IgG, IgM and/or IgA antibodies, due to the serum abundance of said antibodies.

In a further embodiment, the invention provides a method for identifying IgG subclasses of antibodies specific for AD-associated antigen present in a subject, comprising the steps of exposing a biological fluid from the subject (whose biological fluid is believed to comprise an antibody that recognizes an AD-associated antigen and belongs to an Ig class) with the immobilized antigen associated with AD, exposing the biological fluid to a detectable binding agent that is capable of specifically binding antibodies belonging to the IgG subclass, and detecting the detectable moiety for the purpose of identifying the antibody as belonging to the IgG subclass. In a certain embodiments, the antibody of the biological fluid being detected is selected from the group consisting of IgG₁, IgG₂, IgG₃ and IgG₄. In certain embodiments, the binding agent of the invention is capable of binding an antibody Fc region. In certain embodiments, the binding agent of the invention is an antibody. In a exemplary embodiments, the antibody is an anti-IgG antibody, an anti-IgM antibody (e.g., an anti-human IgM Fc_{5μ}, fragment specific antibody), an anti-IgA antibody (e.g., anti-human IgA α-chain specific antibody) or an anti-IgE antibody. In exemplary embodiments, the antibody is an anti-IgG₁ antibody, an anti-IgG₂ antibody, an anti-IgG₃ antibody or an anti-IgG₄ antibody.

In one embodiment, the immobilized antigen comprises β-amyloid or fragment(s) thereof. In a related embodiment, the immobilized antigen of the invention comprises a synthetic 42 amino acid β-amyloid (AN1792) or fragment(s) thereof.

In exemplary embodiments, the binding agent is conjugated to alkaline phosphatase. In additional embodiments, the binding agent is conjugated to a detectable moiety (e.g., a chromophore or an isotope). In other embodiments, the binding agent is fluorescently labeled or radioactively labeled. In a further related embodiment, the detectable moiety of the invention is an enzyme. In another related embodiment, the enzyme is selected from the group consisting of horseradish peroxidase, urease, alkaline phosphatase, glucoamylase and β-galactosidase. In embodiments featuring an enzyme as the detectable moiety, the assay sample is exposed to a substrate, for example a substrate that changes color in the presence of the enzyme, for the purposes of detection.

In exemplary embodiments of the invention, the biological fluid is selected from the group consisting of serum, plasma or cerebrospinal fluid (CSF). In additional embodiments, other biological fluids are used, including, e.g., urine, saliva, nasal discharge, etc.

In order to provide a clear understanding of the specification and claims, the following definitions are conveniently provided below.

### DEFINITIONS

As used herein, the terms “neurodegenerative disorder” or “neurodegenerative disease” refer broadly to disorders or diseases relating to or characterized by degeneration of neurons and/or nervous tissue. The term “amyloidogenic disease” or “amyloidogenic disorder” includes any disease associated with (or caused by) the formation or deposition of insoluble amyloid fibrils. Exemplary amyloidogenic diseases include, but are not limited to systemic amyloidosis, Alzheimer's disease (AD), cerebral amyloid angiopathy (CAA), mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively). Different amyloidogenic diseases are defined or characterized by the nature of the polypeptide component of the fibrils deposited. For example, in subjects or patients having Alzheimer's disease, β-amyloid protein (e.g., wild-type, variant, or truncated β-amyloid protein) is the principal polypeptide component of the amyloid deposit. Accordingly, Alzheimer's disease is an example of a “disease characterized by deposits of Aβ” or a “disease associated with deposits of Aβ”, e.g., in the brain of a subject or patient. Other diseases characterized by deposits of Aβ can include uncharacterized diseases where amyloidogenic deposits are found in one or more regions of the brain associated with learning and/or memory, e.g., the hippocampus, amygdala, subiculum, cingulated cortex, prefrontal cortex, perirhinal cortex, sensory cortex, and medial temporal lobe.

As used herein the terms “neurodegenerative disease-associated antigen”, “NDAA”, and “antigen associated with neurodegenerative disease” refer to an antigen whose existence (e.g., expression, level, and/or activity, etc.) correlates with or relates to the appearance, onset, progression or outcome of a neurodegenerative disease. Similarly, the terms “AD-associated antigen” and “antigen associated with AD” refer to an antigen whose existence (e.g., expression, level, and/or activity, etc.) correlates with or relates to the appearance, onset, progression or outcome of AD. The correlation or relation may be either positive or negative. In preferred embodiments of the instant invention, an “antigen associated with AD” is β-amyloid or a fragment thereof.

The terms “β-amyloid protein”, “β-amyloid peptide”, “β-amyloid”, “Aβ” and “Aβ peptide” are used interchangeably herein. Aβ (e.g., Aβ₃₉, Aβ₄₀, Aβ₄₁, Aβ₄₂ and Aβ₄₃) is a ˜4-kDa peptide of 39-43 amino acids resulting from β-secretase cleavage of the larger transmembrane glycoprotein termed Amyloid Percursor Protein (APP). Multiple isoforms of APP exist, for example APP⁶⁹⁵, APP⁷⁵¹, and APP⁷⁷⁰. Amino acids within APP are assigned numbers according to the sequence of the APP⁷⁷⁰ isoform (see e.g., GenBank Accession No. P05067). Examples of specific isotypes of APP which are currently known to exist in humans are the 695 amino acid polypeptide described by Kang et. al. (1987) Nature 325:733-736 which is designated as the “normal” APP; the 751 amino acid polypeptide described by Ponte et al. (1988) Nature 331:525-527 (1988) and Tanzi et al. (1988) Nature 331:528-530; and the 770-amino acid polypeptide described by Kitaguchi et. al. (1988) Nature 331:530-532. As a result of proteolytic processing of APP by different secretase enzymes in vivo or in situ, Aβ is found in both a “short form”, 40 amino acids in length, and a “long form”, ranging from 42-43 amino acids in length. The short form, Aβ₄₀, consists of residues 672-711 of APP. The long form, e.g., Aβ₄₂ or Aβ₄₃, consists of residues 672-713 or 672-714, respectively. Part of the hydrophobic domain of APP is found at the carboxy end of Aβ, and may account for the ability of Aβ to aggregate, particularly in the case of the long form.

Aβ peptide can be found in or purified from the body fluids of humans and other mammals, e.g. cerebrospinal fluid, including both normal individuals and individuals suffering from amyloidogenic disorders. The terms “β-amyloid protein”, “β-amyloid peptide”, “β-amyloid”, “Aβ” and “Aβ peptide” include peptides resulting from secretase cleavage of APP and synthetic peptides having the same or essentially the same sequence as the cleavage products. A specific form of synthetic β-amyloid peptide used herein is the AN1792 peptide, which constitutes a synthetic Aβ₄₂ fragment.

Aβ peptide also refers to related Aβ sequences that results from mutations in the Aβ region of the normal gene. Examples of specific variants of APP include point mutation which can differ in both position and phenotype (for review of known variant mutation see Hardy (1992) Nature Genet. 1:233-234). All references cited here are incorporated by reference. The term “APP fragments” as used herein refers to fragments of APP other than those which consist solely of β-amyloid protein or β-amyloid protein fragments. That is, APP fragments will include amino acid sequences of APP in addition to those which form intact β-amyloid protein or a fragment of β-amyloid protein.

The term “soluble Aβ” or “dissociated Aβ” refers to the non-aggregating or disaggregated Aβ polypeptide, including monomeric soluble as well as oligomeric soluble Aβ polypeptide (e.g., soluble Aβ dimers, trimers, and the like). Soluble Aβ can be found in vivo in biological fluids such as cerebrospinal fluid and/or serum. Soluble Aβ can also be prepared in vitro, e.g., by solubilizing Aβ peptide in appropriate solvents and/or solutions. For example, soluble Aβ can be prepared by dissolving lyophilized peptide in alcohol, e.g., HFIP followed by dilution into cold aqueous solution. Alternatively, soluble Aβ can be prepared by dissolving lyophilized peptide in neat DMSO with sonication. The resulting solution can be centrifuged (e.g., at 14,000×g, 4° C., 10 minutes) to remove any insoluble particulates.

The term “insoluble Aβ” or “aggregated Aβ” refers to an aggregated Aβ polypeptide, for example, Aβ held together by noncovalent bonds and which can occur in the fibrillary, toxic, β-sheet form of Aβ peptide that is found in neuritic plaques and cerebral blood vessels of patients with AD. Aβ (e.g., Aβ42) is believed to aggregate, at least in part, due to the presence of hydrophobic residues at the C-terminus of the peptide (part of the transmembrane domain of APP).

The term “immunoglobulin” or “antibody” (used interchangeably herein) refers to a protein having a basic four-polypeptide chain structure consisting of two heavy and two light chains, said chains being stabilized, for example, by interchain disulfide bonds, which has the ability to specifically bind antigen. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen.

The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

Immunoglobulins or antibodies can exist in monomeric or polymeric form, for example, IgM antibodies which exist in pentameric form and/or IgA antibodies which exist in monomeric, dimeric or multimeric form. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)₂, Fabc and/or Fv fragments. The term “antigen-binding fragment” refers to a polypeptide fragment of an immunoglobulin or antibody that binds antigen or competes with intact antibody (i.e., with the intact antibody from which they were derived) for antigen binding (i.e., specific binding). Binding fragments are produced by recombinant DNA techniques, or by enzymatic or chemical cleavage of intact immunoglobulins. Binding fragments include Fab, Fab′, F(ab′)₂, Fabc, Fv, single chains, and single chain antibodies. Other than “bispecific” or “bifunctional” immunoglobulins or antibodies, an immunoglobulin or antibody is understood to have each of its antigen-binding sites identical. A “bispecific” or “bifunctional antibody” is an artificial hybrid antibody having two different heavy/light chain pairs and two different antigen-binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

“Specific binding” of an antibody means that the antibody exhibits appreciable affinity for a particular antigen or epitope and, generally, does not exhibit significant crossreactivity. In exemplary embodiments, the antibody exhibits no crossreactivity (e.g., does not crossreact with non-Aβ peptides or with remote epitopes on Aβ). “Appreciable” or preferred binding includes binding with an affinity of at least 10⁶, 10⁷, 10⁸, 10⁹ M⁻¹, or 10¹⁰ M⁻¹. Affinities greater than 10⁷ M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant crossreactivity” is one that will not appreciably bind to an undesirable entity (e.g., an undesirable proteinaceous entity). For example, an antibody that specifically binds to Aβ will appreciably bind Aβ but will not significantly react with non-Aβ proteins or peptides (e.g., non-Aβ proteins or peptides included in plaques). An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody. See, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539 (incorporated by reference in their entirety for all purposes). The assignment of amino acids to each domain is in accordance with the definitions of Kabat, Sequences of Proteins of Immunological Interest (National Institutes of Health, Bethesda, Md., 1987 and 1991). An alternative structural definition has been proposed by Chothia et al., J. Mol. Biol. 196:901 (1987); Nature 342:878 (1989); and J. Mol. Biol. 186:651 (1989) (hereinafter collectively referred to as “Chothia et al.” and incorporated by reference in their entirety for all purposes).

The phrase “substantially from a human immunoglobulin or antibody” or “substantially human” means that, when aligned to a human immunoglobulin or antibody amino sequence for comparison purposes, the region shares at least 80-90%, 90-95%, or 95-99% identity (i.e., local sequence identity) with the human framework or constant region sequence, allowing, for example, for conservative substitutions, consensus sequence substitutions, germline substitutions, backmutations, and the like. The introduction of conservative substitutions, consensus sequence substitutions, germline substitutions, backmutations, and the like, is often referred to as “optimization” of a humanized antibody or chain. The phrase “substantially from a non-human immunoglobulin or antibody” or “substantially non-human” means having an immunoglobulin or antibody sequence at least 80-95%, preferably at least 90-95%, more preferably, 96%, 97%, 98%, or 99% identical to that of a non-human organism, e.g., a non-human mammal.

Accordingly, all regions or residues of a humanized immunoglobulin or antibody, or of a humanized immunoglobulin or antibody chain, except possibly the CDRs, are substantially identical to the corresponding regions or residues of one or more native human immunoglobulin sequences. The term “corresponding region” or “corresponding residue” refers to a region or residue on a second amino acid or nucleotide sequence which occupies the same (i.e., equivalent) position as a region or residue on a first amino acid or nucleotide sequence, when the first and second sequences are optimally aligned for comparison purposes.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species. The terms “humanized immunoglobulin” or “humanized antibody” are not intended to encompass chimeric immunoglobulins or antibodies, as defined infra. Although humanized immunoglobulins or antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

In certain embodiments of the instant invention, “antibodies” or “immunoglobulins” are useful in assays to detect the antigen which stimulated their production (for preferred embodiments of the invention, β-amyloid peptide or a fragment thereof).

As used herein, the term “monoclonal antibody” refers to an antibody derived from a single clone of B lymphocytes (i.e., B cells) which is homogeneous in structure and antigen specificity. As used herein, the term “polyclonal antibody” refers to a plurality of antibodies originating from many different clones of antibody-producing cells which are heterogeneous in their structure and epitope specificity, but all recognize the same antigen. Monoclonal and polyclonal antibodies may exist within bodily fluids, as crude preparations, or may be purified, as in embodiments of the invention involving passive immunization of individuals. It is intended that the term “antibody” encompass any Ig (e.g., IgG, IgM, IgA, IgE, etc.) obtained from any source (e.g., in exemplary embodiments, humans and non-human primates, and in additional embodiments, rodents, lagomorphs, caprines, bovines, equines, ovines, etc.).

The term “biological fluid”, as used herein, refers to any biological matter obtained from a subject existing in a liquid solution or fluid form. The term also refers to any components purified or otherwise extracted from such matter, thus including, for example, plasma derived from blood samples and antigen-binding agents derived from biological fluids.

The term “binding agent”, as used herein, refers to any agent capable of specifically binding to an antibody. In certain embodiments of the invention, the binding agent is itself an antibody, antibody fragment, or construct thereof. The binding agent may also comprise synthetic, modified or naturally-occurring moieties or oligomers capable of specifically recognizing antibodies, including, for example, aptamers, synthetic constructs comprising epitope-recognizing CDRs, etc.

The term “detectable moiety” or “detectable label”, as used herein, refers to a moiety that is attached through covalent or non-covalent means to a binding agent. A “detectable moiety” provides a means for detection or quantitation of the binding agent comprising the detectable moiety. In exemplary embodiments, the detectable moiety is a calorimetric moiety. In additional embodiments, the “detectable moiety” can be a radioactive moiety, a fluorescent moiety, a chemiluminescent moiety, a mass label, a charge label, an enzyme (e.g. for which substrate converting activity of the enzyme is observed to reveal presence of the binding agent), etc.

The term “fluorescent moiety” refers to a label that accepts radiant energy of one wavelength and emits radiant energy of a second wavelength.

The term “Ig class” or “immunoglobulin class”, as used herein, refers to the five classes of immunoglobulin that have been identified in humans and higher mammals, IgG, IgM, IgA, and IgE.

The term “Ig subclass” refers to the two subclasses of IgM (H and L) and four subclasses of IgG (IgG₁, IgG₂, IgG₃, and IgG₄) that have been identified in humans and higher mammals. The term “IgG subclass” refers to the four subclasses of immunoglobulin class IgG—IgG₁, IgG₂, IgG₃, and IgG₄ that have been identified in humans and higher mammals by the γ heavy chains of the immunoglobulins, γ₁-γ₄, respectively.

An “antigen” is an entity (e.g. a proteinaceous entity or peptide) to which an immunoglobulin or antibody specifically binds. The terms “antigen fragment” and “portion of an antigen” and the like are used in reference to a portion of an antigen. Antigen fragments or portions typically range in size, from a small percentage of the entire antigen to a large percentage, but not 100%, of the antigen. Antigen fragments and/or portions thereof may or may not comprise an “epitope” recognized by an antibody, and also may or may not be immunogenic in an individual or population.

The term “epitope” or “antigenic determinant” refers to a site on an antigen to which an immunoglobulin or antibody (or antigen binding fragment thereof) specifically binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

The term “immunological” or “immune” response is the development of a humoral (antibody mediated) and/or a cellular (mediated by antigen-specific T cells or their secretion products) response directed against an antigen in a subject. Such a response can be an active response induced by administration of immunogen or a passive response induced by administration of antibody or primed T-cells. A cellular immune response is elicited by the presentation of polypeptide epitopes in association with Class I or Class II MRC molecules to activate antigen-specific CD4⁺ T helper cells and/or CD8⁺ cytotoxic T cells. The response may also involve activation of monocytes, macrophages, natural killer (“NK”) cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays (see Burke, REF; Tigges, REF). The relative contributions of humoral and cellular responses to the protective or therapeutic effect of an immunogen can be distinguished by separately isolating antibodies and T-cells from an immunized animal or individual and measuring protective or therapeutic effect in a second subject.

As used herein, the term “immunotherapy” refers to a treatment, for example, a therapeutic or prophylactic treatment, of a disease or disorder intended to and/or producing an immune response (e.g., an active or passive immune response).

An “immunogenic agent” or “immunogen” is capable of inducing an immunological response against itself on administration to a patient, optionally in conjunction with an adjuvant. An “immunogenic composition” is one that comprises an immunogenic agent.

The term “adjuvant” refers to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages.

As used herein, the term “ELISA” refers to enzyme-linked immunosorbent assay (or EIA). In certain embodiments of the invention, an “indirect ELISA” is used. In select embodiments, an antigen (or antibody) is immobilized to a solid support (e.g., a microtiter plate well), and is detected indirectly by first adding a sample that might contain an antigen-specific antibody, then followed by the addition of a detection binding agent or antibody specific for the antibody that specifically binds the antigen. In preferred embodiments of the present invention, these secondary binding agents or antibodies specifically recognize the Ig class or subclass of the primary antibodies. Such secondary binding agents or antibodies may be “species-specific” antibodies (e.g., a goat anti-rabbit antibody), which are available from various manufacturers known to those in the art (e.g., Santa Cruz Biotechnology; Zymed; and Pharmingen/Transduction Laboratories). ELISA methods and applications are known in the art, and are described in several references (See, e.g., Crowther, “Enzyme-Linked Immunosorbent Assay (ELISA),” in Molecular Biomethods Handbook, Rapley et al. [eds.], pp. 595-617, Humana Press, Inc., Totowa, N.J. [1998]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]).

As used herein, the term “signal” is used generally in reference to any detectable process that indicates that a reaction has occurred, for example, binding of antibody to antigen. It is contemplated that signals in the form of radioactivity, fluorimetric or colorimetric products/reagents will all find use with the present invention. In various embodiments of the present invention, the signal is assessed qualitatively, while in alternative embodiments, the signal is assessed quantitatively.

As used herein, the term “solid support” is used in reference to any solid or stationary material to which reagents such as antibodies, antigens, and other test components are attached. For example, in an ELISA method, the wells of microtiter plates may provide solid supports. Other examples of solid supports include chips, membranes, frits, slides, plates, coverslips, beads, particles, cell culture flasks, as well as many other suitable items. In select embodiments of the invention, the substrate of the solid support comprises polystyrene, controlled-pore-glass, glass, silica gel, silica, polyacrylamide, magnetic beads, polyacrylate, hydroxyethylmethacrylate, polyaminde, polyethylene, polyethyleneoxy, and copolymers and grafts of any of the above solid substrates.

The term “patient” includes human and other mammalian subjects that receive either prophylactic or therapeutic treatment. Exemplary patients receive either prophylactic or therapeutic treatment with the immunotherapeutic agents of the invention.

As used herein, the term “kit” is used in reference to a combination of reagents and other materials which facilitate sample analysis. In some embodiments, the immunoassay kit of the present invention includes a suitable antigen, binding agent comprising a detectable moiety, and detection reagents. A system for amplifying the signal produced by detectable moieties may or may not also be included in the kit. Furthermore, in other embodiments, the kit includes, but is not limited to, components such as apparatus for sample collection, sample tubes, holders, trays, racks, dishes, plates, instructions to the kit user, solutions or other chemical reagents, and samples to be used for standardization, normalization, and/or control samples.

As used herein, the term “in vitro” refers to an artificial environment and to processes or reactions that occur within an artificial environment. In vitro environments consist of, but are not limited to, controlled laboratory conditions. The term “in vivo” refers to the natural environment (e.g., an animal or a cell) and to processes or reactions that occur within that natural environment.

Various methodologies of the instant invention include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing a methodology of the invention, as described herein. For example, the level and classes of anti-β-amyloid antibody in one sample can be determined prior to assessing the level and classes of anti-β-amyloid antibody in a test sample. In another embodiment, a “suitable control” or “appropriate control” is a value, level, feature, characteristic, property, etc. determined in a subject, e.g., a control or normal subject exhibiting, for example, normal traits. In yet another embodiment, a “suitable control” or “appropriate control” is a predefined value, level, feature, characteristic, property, etc.

ELISA Assay Formats

The invention provides methods for performing Ig profiling and titer assessment with high levels of sensitivity, specificity and precision in subjects, for example, in patients receiving an AD immunotherapy. The assays of the invention involve determining the levels of an anti-Aβ antibody produced, for example, in a patient receiving the AD therapy. Levels of anti-Aβ antibody are determined by in a biological sample from the patient using an Aβ antigen to bind the antibodies from the sample. In exemplary formats, the Aβ antigen is present in a sufficient amount to bind at least 80%, 90%, 95% or more, or 100% of the Aβ antibodies in the biological sample. In such formats, the antigen is present at a level that exceeds the level of Aβ antibodies (i.e., is in excess of the level of antibodies present). Detection of the bound antibodies is performed. Certain assay formats feature detection of antibodies using an additional antibody (or additional antibodies). Additional antibodies (e.g., second or third antibodies, referred to alternatively as secondary or tertiary antibodies, respectively) can be detectably labeled. Labeling antibodies with an enzyme, the enzyme having a detectable activity, is featured.

In certain embodiments (described in detail in the Examples herein), a synthetic Aβ₄₂ peptide, AN1792, is used as the antigen. Use of such an Aβ antigen allows for highly specific and sensitive detection of antibodies that recognize the Aβ₄₂ peptide. The level of sensitivity and specificity of the methods of the instant invention can be demonstrated, for example, in competitive inhibition assays, as detailed in the Examples. This high level of sensitivity and specificity is especially important during performance of subclass ELISA, when traditionally during immune responses to immunogenic compositions very low levels of IgG₄ are generated. Specificity of the ELISAs can be confirmed, for example, by examining binding reagents used in each ELISA for the ability to bind analytes (e.g., antigens) of interest without significant binding to other analytes. Sensitivity can be confirmed, for example, by determining the ability of binding reagents produce a significant signal or readout (e.g., colorometric signal or readout) at a significant dilution of sample, for example, at a dilution of 1:200, 1:100 or 1:50. The reciprocal of a dilution at which a significant signal or readout is obtained is also referred to as a titer.

Such antibody titers can also be expressed in terms of concentration of antibody present in the biological sample e.g., in μg/ml. Such measurements are easily achieved using a quantifiable control or standard, e.g., a purified immunoglobulin or antibody sample. Measurement of antibody titers can be expressed, for example, in ranges from, e.g., 0.01 pg/ml to 100 mg/ml, and all discrete numerical values within this range. Antibody concentration can therefore also be expressed as e.g., greater than 0.01 pg/ml, greater than 0.1 pg/ml, greater than 1 pg/ml, greater than 10 pg/ml, greater than 100 pg/ml, greater than 1 μg/ml, greater than 10 μg/ml, greater than 100 μg/ml, greater than 1 mg/ml, and greater than 10 mg/ml. Ranges within the above-recited values are also intended to be included in the scope of the instant invention, e.g., 100 pg/ml-1 μg/ml, 1 μg/ml-10 μg/ml, and the like

The methods of the instant invention are also highly precise, as can be documented, e.g., by assessing both within day and between day titer measurements performed with both control sera or purified antibodies (e.g., monoclonal antibodies). Precision can also be referred to as accuracy or exactness and is the degree to which the assay reproduces between assays, for example, performed on the same day using different reagents or my different operators, or performed on different days. The assays of the instant invention are precise for example, varying less than 20%, less than 10%, or less than 5% between assays. In certain embodiments, precision is within 1-5%, 5-10%, 10-15%, or 15-20%. Methods of determining inter-assay variability measurements are described in greater detail in the Examples. Precision can be indicated as a % CV (% Coefficient of Variability).

In an additional embodiment, assay results from test subjects are compared to assay results from suitable controls.

In certain embodiments, the amount of antibody that recognizes an antigen in a test sample is quantitatively measured. In related embodiments, the amount of antibody that recognizes an antigen in a test sample is quantitatively measured via comparison with an appropriate internal control.

In some embodiments, the amount of antibody that recognizes an antigen in a test sample is quantitated via normalization to the amount of antibody in a monkey sera control that recognizes the antigen. In some embodiments, a sera control is used as an internal control. Sera from monkeys immunized with AN1792 is an exemplary control for Ig class-specific assays as such sera is known to include, for example, high levels of IgG antibodies can react with such monkey antibodies (i.e., cross-react). For example, monkey sera IgG can exhibit about 85-95% cross-reactivity with anti-human polyclonal antibodies (Monkey sera IgM and IgA can additionally exhibit approx. 50% and 20% cross-reactivity with anti-human polyclonal antibodies, respectively). Such sera is referred to as a positive control. Additional positive controls include, but are not limited to purified recombinant antibodies, for example, chimeric antibodies or humanized antibodies. In certain embodiments, test samples are compared to a positive control sample having a known or predetermined value. For example, a serum known to contain significant levels of Aβ antibodies (known, for example, from repeated assaying of said samples for the presence of such antibodies) can be assigned a value in certain units, for example, in ELISA units or “EU”. AN1792 monkey sera (or pooled sera) can be assigned a value or 100 EU/ml for the level of IgG antibody. A single dilution (or several dilutions) of such a sample can be included in each assay for control or standardization. Controls are included at a dilution that results in a value of, for example, from 50-70 EU (e.g., a value within the reportable range of the assays). Test samples (e.g., biological samples from patients) can be compared to such internal controls. For example, test samples can have between 20 and an infinite EU measurement as compared to the control (no upper range may be placed on the range of EU detection, as concentrated samples may be diluted prior to detection of EU values). For Ig subclass specific assays, sera from a patient (e.g., a human patient being treated with an AD immunotherapy) having a known or predetermined level of a particular Ig subclass can be used as a positive control. Alternatively, human patient sera can be pooled. Alternatively, a universal positive control can be made by pooling sera having high levels, either alone or combined, of each IgG subclass.

In certain embodiments, assay results from test subjects are compared to assay results obtained from control samples derived from normal healthy adult individuals. In a related embodiment, assay results from test subjects are compared to assay results obtained from control samples derived from individuals of less than 18 years of age. In another related embodiment, assay results from test subjects are compared with assay results obtained from control samples derived from individuals of less than 4 years of age. The latter controls are often referred to as “negative” controls as these sera do not usually contain any anti-Aβ antibodies. Inclusion of negative controls helps to eliminate the possibility of a test sample giving a falsely positive signal or readout (i.e., “false positives”).

An exemplary assay format for determining the level of an anti-Aβ antibody of a particular Ig class is as follows. A biological sample from a patient (e.g., a serum or CSF sample) is exposed to immobilized Aβ₁₋₄₂ (e.g., AN1792), for example, immobilized on the surface of the wells of a microtiter plate. The sample is exposed to the immobilized antigen under conditions sufficient for binding of the antigen to Aβ antibodies present in the sample. The antigen is preferably in excess of the antibody. Components of the biological sample which do not bind antigen (i.e., non-anti-Aβ antibody components) are washed from the wells. The bound anti-Aβ antibodies are exposed to an Ig class-specific antibody. The Ig class-specific antibody is conjugated to an enzyme, for example, a polyclonal antibody which specifically recognizes the Ig class (e.g., an anti-IgG, anti-IgA, anti-IgM or anti-IgE antibody). Such polyclonal antibodies are, for example, goat or rabbit antibodies. An exemplary enzyme is alkaline phosphatase (AP). Detection of AP is accomplished via exposure of the enzyme to p-nitrophenyl phosphate (pNPP), a substrate producing a colorimetric change upon cleavage of a phosphate group by the AP enzyme. Such calorimetric change is observed by detecting the optical density (OD) of the sample at an appropriate wavelength (e.g., 405 nM). An ELISA standard is included in exemplary assays. The standard can be a human or mammalian sera sample diluted such that an OD₄₀₅ of about 0.3 is obtained. Test samples can be normalized to such an internal control, for example, to reduce day-to-day differences which may result, for example, from daily fluctuations in room temperature, humidity and the like, or differences which may result from different preparations or reagents (e.g., buffers, detection reagents and the like). Positive and/or negative control sera (i.e., sera for which a predetermined positive or negative signal or output is known) can be included to ensure that the assay is performing as expected. Test sera can be assayed at multiple dilutions, for example, at serial dilutions of 1:200, 1:100, 1:50, 1:25, 1:12.5, etc.

An exemplary assay format for determining the level of an anti-Aβ antibody of a particular Ig subclass is as follows. A biological sample from a patient (e.g., a serum or CSF sample) is exposed to immobilized Aβ₁₋₄₂ (e.g., AN1792), for example, immobilized on the surface of the wells of a microtiter plate. The sample is exposed to the immobilized antigen under conditions sufficient for binding of the antigen to anti-Aβ antibodies present in the sample. The antigen is preferably in excess of the antibody. Components of the biological sample which do not bind antigen (i.e., non-anti-Aβ antibody components) are washed from the wells. The bound anti-Aβ antibodies (primary antibodies) are exposed to an Ig subclass-specific antibody (a specific secondary antibody), for example, an anti-human IgG₁ antibody, an anti-human IgG₂ antibody, an anti-human IgG₃ antibody or an anti-human IgG₄ antibody. Ig subclass-specific monoclonal antibodies are exemplary. The secondary antibody may be conjugated to an enzyme, for example, a polyclonal antibody which specifically recognizes the secondary antibody. Such polyclonal antibodies are, for example, goat or rabbit antibodies. Exemplary detection is via an enzyme (e.g., Aβ ) and colorimetric substrate (e.g., pNPP), as described above. An exemplary standard is a human or mammalian sera sample diluted such that an OD₄₀₅ of about 0.3 is obtained. Test samples can be normalized to such an internal control. Positive control sera include human sera known or predetermined to contain a significant level of one or more Ig subclasses. Negative control sera are as described above. Test sera can be assayed at multiple dilutions, for example, at dilutions of 1:50,.1:25, 1:12.5, etc.

The levels of various Ig class and/or subclass antibodies can be used as an indicator of desirable and/or undesirable physiological responses in a patient being treated with an AD immunotherapy. For example, elevated levels of Aβ antibodies of the IgG and/or IgA Ig class can indicate a positive response to the administration of an Aβ immunogenic composition, for example, an immunogenic composition comprising AN1792. Moreover, elevated levels Aβ antibodies of the IgG1 and/or IgG2 Ig subclass can indicate a positive response to the administration of an Aβ immunogenic composition, for example, an immunogenic composition comprising AN1792. Moreover, it has been recently demonstrated that the encephalitis observed in certain patients receiving an AN1792 immunogenic composition was primarily a Th1 immune response rather than a Th2 response. Accordingly, elevated levels of, for example, IgG2 levels as compared to IgG1 levels or IgG versus IgE levels may indicate an undesirable Th1 response and may be relied on by a skilled physician to alter the course of AD immunogenic composition. When determining the level of more than one Ig class or subclass of Aβ antibodies, the levels of such antibodies can be compared, for example, as a ratio where the levels of two antibodies classes and/or subclasses are being determined, or as a profile where the levels of two, three, four, five or more antibodies classes and/or subclasses are being determined. Levels, ratios or profiles of anti-Aβ antibody classes and/or subclasses can be used with any other indicator of a patient's immune response or physical state in determining an appropriate course of AD immunotherapy. For example, an anti-Aβ antibody class and/or subclass level, ration or profile can be used with other Th1/Th2 indicators (e.g., gene chips, cellular activation and/or proliferation assays, for example, T cell assays, cytokine assays, and the like) to determine or alter a course of treatment.

Immunological and Therapeutic Reagents

Immunological and therapeutic reagents of the invention comprise or consist of immunogens or antibodies, or functional or antigen binding fragments thereof, as defined herein. The basic antibody structural unit (e.g., of a human or humanized antibody) is known to comprise a tetramer of subunits. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as either kappa or lambda and are about 230 residues in length. Heavy chains are classified as gamma (γ), mu (μ), alpha (α), delta (δ), or epsilon (ε), are about 450-600 residues in length, and define the antibody's isotype as IgG, IgM, IgA and IgE, respectively. Additionally, humans have been found to have four subclasses of IgG (IgG₁, IgG₂, IgG₃ and IgG₄) and two subclasses of IgA (IgA₁ and IgA₂). The present invention particularly concerns subclasses of IgGs. Thus, as used herein and in the claims, the term “IgG” is meant to refer to any IgG, including but not limited to IgGs from any warm-blooded vertebrate subject, and including but not limited to polyclonal IgGs and monoclonal IgGs. As is recognized among those having ordinary skill in the art, IgGs, including warm-blooded vertebrate IgGs, may be divided into subclasses, including but not limited to IgG₁, IgG₂, IgG₃ and IgG₄. Thus, the term “IgG” is also meant to include all subclasses of IgGs.

Both heavy and light chains of an antibody are folded into domains. The term “domain” refers to a globular region of a protein, for example, an Ig or antibody. Ig or antibody domains include, for example, three or four peptide loops stabilized by β-pleated sheet and an interchain disulfide bond. Intact light chains have, for example, two domains (V_{L} and C_{L}) and intact heavy chains have, for example, four or five domains (V_{H}, C_{H}1, C_{H}2, and C_{H}3).

Within light and heavy chains, the variable and constant regions are joined by a “J” region of about 12 or more amino acids, with the heavy chain also including a “D” region of about 10 more amino acids. (See generally, Fundamental Immunology (Paul, W., ed., 2nd ed. Raven Press, N.Y. (1989), Ch. 7, incorporated by reference in its entirety for all purposes).

The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. Naturally-occurring chains or recombinantly produced chains can be expressed with a leader sequence which is removed during cellular processing to produce a mature chain. Mature chains can also be recombinantly produced having a non-naturally occurring leader sequence, for example, to enhance secretion or alter the processing of a particular chain of interest.

The CDRs of the two mature chains of each pair are aligned by the framework regions, enabling binding to a specific epitope. From N-terminal to C-terminal, both light and heavy chains comprise the domains FR1, CDR1, FR2, CDR2, FR3, CDR3 and FR4. “FR4” also is referred to in the art as the D/J region of the variable heavy chain and the J region of the variable light chain. The assignment of amino acids to each domain is in accordance with the definitions of Kabat, Sequences of Proteins of Immunological Interest (National Institutes of Health, Bethesda, Md., 1987 and 1991). An alternative structural definition has been proposed by Chothia et al., J. Mol. Biol. 196:901 (1987); Nature 342:878 (1989); and J. Mol. Biol. 186:651 (1989) (hereinafter collectively referred to as “Chothia et al.” and incorporated by reference in their entirety for all purposes).

β-Amyloid Antibodies

Methods and kits of the invention include antibodies that specifically bind to β-amyloid or other components of amyloid plaques. Such antibodies can be monoclonal or polyclonal. Some such antibodies bind specifically to the aggregated form of β-amyloid without binding to the soluble form. Some bind specifically to the soluble form without binding to the aggregated form. Some bind to both aggregated and soluble forms. Some such antibodies bind to a naturally occurring short form of β-amyloid (i.e., Aβ39, 40 or 41) without binding to a naturally occurring long form of β-amyloid (i.e., Aβ42 and Aβ43). Some antibodies bind to a long form of β-amyloid without binding to a short form. Some preferred antibodies bind to β-amyloid without binding to full-length amyloid precursor protein. Exemplary antibodies used in therapeutic methods have an intact constant region or at least sufficient of the constant region to interact with an Fc receptor. Human isotype IgG1 is often used because of it having highest affinity of human isotypes for the FcRI receptor on phagocytic cells. Bispecific Fab fragments can also be used, in which one arm of the antibody has specificity for β-amyloid, and the other for an Fc receptor. Preferred antibodies bind to β-amyloid with a binding affinity greater than (or equal to) about 10⁶, 10⁷, 10⁸, 10⁹, or 10¹⁰ M⁻¹ (including affinities intermediate of these values).

Polyclonal sera typically contain mixed populations of antibodies binding to several epitopes along the length of β-amyloid. However, polyclonal sera can be specific to a particular segment of β-amyloid, such as β-amyloid 1-10. Monoclonal antibodies bind to a specific epitope within Aβ that can be a conformational or nonconformational epitope. Prophylactic and therapeutic efficacy of antibodies can be tested using transgenic animal model procedures prior to administration to human subjects. Exemplary epitopes or antigenic determinants to which an antibody of the invention binds can be found within the human amyloid precursor protein (APP), but are preferably found within the Aβ peptide of APP. Exemplary epitopes or antigenic determinants, as described herein, are located within the N-terminus of the Aβ peptide and include residues within amino acids 1-10 of Aβ, preferably from residues 1-3, 1-4, 1-5, 1-6, 1-7 or 3-7 of Aβ42. Other exemplary epitopes or antigenic determinants start at residues 1-3 and end at residues 7-11 of Aβ. Other exemplary epitopes or antigenic determinants comprise residues 10-15, 15-20, 25-30, 10-20, 20-30, or 10-25 of Aβ. Additional exemplary epitopes or antigenic determinants include residues 2-4, 5, 6, 7 or 8 of Aβ, residues 3-5, 6, 7, 8 or 9 of Aβ, or residues 4-7, 8, 9 or 10 of Aβ42. Such epitopes can be referred to as N-terminal epitopes. Additional exemplary epitopes or antigenic determinants include residues 19-22, 23 or 24 of Aβ42. Other exemplary epitopes or antigenic determinants include residues 10-18, 16-24, 18-21 and 33-42 of Aβ42. Additional exemplary epitopes or antigenic determinants include residues 16-21, 22, 23 or 24 of Aβ42. Such epitopes can be referred to as central epitopes. Additional exemplary epitopes or antigenic determinants include residues 33-40 or 33-42 of Aβ. Such epitopes can be referred to as C-terminal epitopes.

An anti-Aβ antibody may also be “C-terminus-specific.” As used herein, the term “C terminus-specific” means that the antibody specifically recognizes a free C-terminus of an Aβ peptide. Examples of C terminus-specific Aβ antibodies include those that: recognize an Aβ peptide ending at residue 40 but do not recognize an Aβ peptide ending at residue 41, 42, and/or 43; recognize an Aβ peptide ending at residue 42 but do not recognize an Aβ peptide ending at residue 40, 41, and/or 43; etc. An anti-Aβ antibody may also be end-specific. As used herein, the term “end-specific” refers to an antibody which specifically binds to the N-terminal or C-terminal residues of an of Aβ peptide but that does not recognize the same residues when present in a longer of Aβ species or in APP. It is recommended that such antibodies be screened for activity in mouse models before use. In some methods, multiple monoclonal antibodies having binding specificities to different epitopes can be used. Such antibodies can be administered sequentially or simultaneously. Antibodies to amyloid components other than β-amyloid can also be used (e.g., administered or co-administered). For example, antibodies can be directed to the amyloid associated protein synuclein.

When an antibody is said to bind to an epitope within specified residues, such as β-amyloid 1-5 for example, what is meant is that the antibody specifically binds to a polypeptide containing the specified residues (i.e., β-amyloid 1-5 in this an example). Such an antibody does not necessarily contact every residue within β-amyloid 1-5. Nor does every single amino acid substitution or deletion with in β-amyloid 1-5 necessarily significantly affect binding affinity. Epitope specificity of an antibody can be determined, for example, by forming a phage display library in which different members display different subsequences of β-amyloid. The phage display library is then selected for members specifically binding to an antibody under test. A family of sequences is isolated. Typically, such a family contains a common core sequence, and varying lengths of flanking sequences in different members. The shortest core sequence showing specific binding to the antibody defines the epitope bound by the antibody. Antibodies can also be tested for epitope specificity in a competition assay with an antibody whose epitope specificity has already been determined. For example, antibodies that compete with the anti-Aβ antibody for binding to β-amyloid bind to the same or similar epitope as said antibody. Screening antibodies for epitope specificity is a useful predictor of therapeutic efficacy.

Monoclonal or polyclonal antibodies that specifically bind to a preferred segment of β-amyloid without binding to other regions of β-amyloid have a number of advantages relative to monoclonal antibodies binding to other regions or polyclonal sera to intact β-amyloid. First, for equal mass dosages, dosages of antibodies that specifically bind to preferred segments contain a higher molar dosage of antibodies effective in clearing amyloid plaques. Second, antibodies specifically binding to preferred segments can induce a clearing response against amyloid deposits without inducing a clearing response against intact APP polypeptide, thereby reducing the potential side effects.

Monitoring Immune Responses

The instant invention at least in part provides methods of identifying components of an immune response against an NDAA, and in specific embodiments, the immune response is raised against β-amyloid peptide in a patient suffering from or susceptible to AD. The methods are particularly useful for monitoring a course of treatment being administered to a patient. The methods can be used to monitor both therapeutic treatment on symptomatic patients and prophylactic treatment on asymptomatic patients. The methods are useful for monitoring both active immunization (e.g., antibody produced in response to administration of immunogen) and passive immunization (e.g., measuring level of administered antibody).

1. Active Immunization

Some methods entail determining a baseline value of an immune response in a patient before administering a dosage of agent, and comparing this with a value for the immune response after treatment. A significant increase (i.e., greater than the typical margin of experimental error in repeat measurements of the same sample, expressed as one standard deviation from the mean of such measurements) in value of the immune response can signal a positive treatment outcome (i.e., that administration of the agent has achieved or augmented an immune response), especially if a specific Ig class(es) or subclass(es) is elevated absolutely or relative to other Ig classes or subclasses. If the absolute level of immune response does not change significantly, or decreases, a negative treatment outcome is indicated. Specific profiles of immunoglobulins in a sample may also indicate a negative outcome that would otherwise have escaped detection in the absence of knowledge of the Ig classes of antibodies present in a sample. In general, patients undergoing an initial course of treatment with an immunogenic agent are expected to show an increase in immune response with successive dosages, which eventually reaches a plateau. Administration of agent is generally continued while the immune response is increasing. Attainment of the plateau is an indicator that the administration of treatment can be discontinued or reduced in dosage or frequency.

In other methods, a control value (i.e., a mean and standard deviation of total anti-NDAA antibody levels and levels of specific Ig classes and subclasses of anti-NDAA antibodies) of immune response is determined for a control population. Typically the individuals in the control population have not received prior treatment. Measured values and Ig profiles of immune response in a patient after administering a therapeutic agent are then compared with the control value. A significant increase in total levels of anti-NDAA antibody relative to the control value (e.g., greater than one standard deviation from the mean) signals a positive treatment outcome, as can significant relative or absolute enhancement of specific Ig classes or subclasses of anti-NDAA antibodies. A lack of significant increase or a decrease in such antibodies signals a negative treatment outcome, as can a relative decrease or lack of response of a given class(es) or subclass(es) of Ig. Administration of agent is generally continued while the immune response is increasing relative to the control value. As before, attainment of a plateau relative to control values is an indicator that the administration of treatment can be discontinued or reduced in dosage or frequency.

In other methods, a control value of immune response (e.g., a mean and standard deviation of total antibody levels and levels of specific Ig classes and subclasses of anti-NDAA antibodies) is determined from a control population of individuals who have undergone treatment with a therapeutic agent and whose immune responses have plateaued in response to treatment. Measured values of immune response in a patient are compared with the control value. If the measured level and Ig profile of response to an NDAA in a patient is not significantly different (e.g., more than one standard deviation) from the control value, treatment can be discontinued. If the level in a patient is significantly below the control value or the Ig profile differs from control profiles, continued administration of agent may be warranted. If the total level of anti-NDAA antibodies or the Ig profile of the patient's anti-NDAA antibodies persists below or is significantly altered from control values, then a change in treatment regimen—for example, use of a different adjuvant—may be indicated.

In other methods, a patient who is not presently receiving NDAA treatment but has undergone a previous course of treatment is monitored for immune response to determine whether a resumption of treatment is required. The measured value and Ig profile of anti-NDAA immune response in the patient can be compared with a value of immune response and Ig profile previously achieved in the patient after a previous course of treatment. A significant decrease in level or alteration of Ig profile of anti-NDAA antibody relative to the previous measurement (e.g., greater than a typical margin of error in repeat measurements of the same sample) can indicate that treatment should be resumed. Alternatively, the anti-NDAA antibody level and Ig profile values measured in a patient can be compared with control values (mean plus standard deviation) determined in a population of patients after undergoing a course of treatment. Alternatively, the measured anti-NDAA antibody and Ig profile values in a patient can be compared with a control value in populations of prophylactically treated patients who remain free of symptoms of disease, or populations of therapeutically treated patients who show amelioration of disease characteristics. In all of these cases, a significant decrease in anti-NDAA antibody level relative to the control level (e.g., more than one standard deviation, and in certain embodiments, more then two standard deviations, or more than four standard deviations) can signal that treatment should be resumed in a patient, while a change in Ig profile of a patient's immune response can also signal the need for resumption, alteration or discontinuation of treatment.

The biological sample for analysis is typically blood, plasma, serum, mucous or cerebrospinal fluid from the patient. The sample is analyzed for indication of an immune response (and classification of Ig profile of the immune response) to any form of NDAA peptide, and in specific embodiments the β-amyloid peptide, typically Aβ₄₂ (AN1792). The immune response (and classification of Ig profile of the immune response) can be determined via identification of, e.g., antibodies that specifically bind to the NDAA. ELISA methods of identifying the Ig profile of antibodies specific to Aβ are described in the Examples section.

2. Passive Immunization

In general, the procedures for monitoring passive immunization are similar to those for monitoring active immunization described above. However, the antibody response following passive immunization typically shows an immediate peak in antibody concentration followed by an exponential decay. Without a further dosage, the decay approaches pretreatment levels within a period of days to months depending on the half-life of the antibody administered. For example the half-life of some human antibodies is of the order of 20 days. Moreover, antibody levels can begin to decrease approximately 1 week after administration.

In some methods, a baseline measurement of anti-NDAA antibody in the patient is made before administration, a second measurement is made soon thereafter to determine the peak antibody level, and one or more further measurements are made at intervals (e.g., daily or weekly) to monitor decay of antibody levels. When the level of antibody has declined to baseline or a predetermined percentage of the peak less baseline (e.g., 50%, 25% or 10%), administration of a further dosage of antibody is performed. In some methods, peak or subsequent measured levels less background are compared with reference levels previously determined to constitute a beneficial prophylactic or therapeutic treatment regime in other patients. If the measured antibody level is significantly less than a reference level (e.g., less than the mean minus one standard deviation of the reference value in population of patients benefiting from treatment) administration of an additional dosage of antibody is indicated. Ig profiles of anti-NDAA antibodies can also be used to dictate dosage timing and alteration of treatment regimens for such passive immunization approaches.

Diagnostic Kits

The invention further provides diagnostic kits for performing the Ig profiling methods of the instant invention. Typically, such kits contain an immobilized antigen to which specific anti-NDAA antibodies bind, one or more anti-NDAA antibodies, and a means for detecting the anti-NDAA antibodies. Such means can include a detectable agent conjugated directly to the anti-NDAA antibody. Alternatively, the means for detecting can include a second (e.g., secondary) antibody which specifically binds to the anti-NDAA antibody conjugated to a detectable agent. Alternatively, the means for detecting can include a second (e.g., secondary) antibody which specifically binds to the anti-NDAA antibody and a third (e.g., tertiary) antibody that specifically binds to the second or secondary antibody conjugated to a detectable agent or moiety. The kit may also provide components useful for detection of the detectable moiety. For detection of antibodies, the NDAA can be supplied prebound to a solid phase, such as to the wells of a microtiter dish.

An additional aspect of the invention provides a kit for prognosticating or monitoring Aβ therapy in a subject, comprising, an antigen associated with Aβ and a detectable binding agent (e.g., antibody) that is capable of specifically binding antibodies that bind the antigen associated with AD. Optionally, the kit(s) include suitable controls. In another embodiment, the invention provides a kit for identifying the Ig class of an antibody associated with AD present in a biological fluid of a subject, comprising an antigen associated with AD and a detectable binding agent (e.g., antibody). An additional embodiment of the invention provides a kit for monitoring a subject treated with an antibody against AD, comprising an antigen associated with AD and a detectable binding agent (e.g., antibody) capable of specifically binding antibodies of the Ig class (e.g., IgG, IgM, IgA and/or IgE) that bind the antigen associated with AD. In another embodiment, the invention provides a kit for identifying IgG subclasses of antibodies present in a subject that are specific for AD-associated antigen, comprising an antigen associated with AD and a detectable binding agent (e.g., antibody) capable of specifically binding antibodies of the IgG subclass (e.g., IgG₁, IgG₂, IgG₃, and/or IgG₄) that bind the antigen associated with AD. In certain embodiments, the AD-associated antigen of the kit comprises β-amyloid or fragment(s) thereof. In other embodiments, the AD-associated antigen of the kit comprises a synthetic 42 amino acid β-amyloid (AN1792) or fragment(s) thereof.

In an additional embodiment, the antibody that binds the AD-associated antigen of the kit is capable of binding an Fc region of said antibody.

In another embodiment, a binding agent of the kit is an antibody (e.g., a second or secondary antibody) selected from the group consisting of an anti-IgG antibody (e.g., an anti-human IgG antibody), an anti-IgM antibody (e.g., an anti-human IgM Fc_{5μ}, fragment specific antibody) and anti-IgA antibody (anti-human IgA a-chain specific antibody). In an additional embodiment, a binding agent of the kit is an antibody selected from the group consisting of an anti-IgG₁ antibody, an anti-IgG₂ antibody, an anti-IgG₃ antibody, and an anti-IgG₄ antibody.

In one embodiment, the detectable moiety of the binding agent of the kit is a chromophore or an isotope. In a related embodiment, the binding agent is fluorescently labeled. In an additional embodiment, the binding agent is radioactively labeled. In a further embodiment, the detectable moiety of the binding agent of the kit is an enzyme. In a related embodiment, the enzyme of the kit is selected from the group consisting of horseradish peroxidase, urease, alkaline phosphatase, glucoamylase and β-galactosidase.

In another embodiment, the kit of the invention further comprises a solid support. In an exemplary embodiment, polystyrene is used as a solid substrate support. Such solid supports include, but are not limited to, solid substrates, chips, beads, small particles, membranes, frits, slides and plates. Solid substrate supports include, but are not limited to, polystyrene, controlled-pore-glass, glass, silica gel, silica, polyacrylamide, magnetic beads, polyacrylate, hydroxyethylmethacrylate, polyamide, polyethylene, polyethyleneoxy, and copolymers and grafts of any of the above solid substrates.

Kits also typically contain labeling providing directions for use of the kit. The labeling may also include a chart or other correspondence regime correlating levels of measured label with levels of anti-NDAA antibodies. The term “labeling” refers to any written or recorded material that is attached to, or otherwise accompanies a kit at any time during its manufacture, transport, sale or use. For example, the term labeling encompasses advertising leaflets and brochures, packaging materials, instructions, audio or video cassettes, computer discs, as well as writing imprinted directly on kits.

Other Applications

The methods of the invention are applicable to a variety of uses including research and diagnostic applications. For example, they are useful for any research application in which an analysis must be performed rapidly or on limited amounts of a sample that potentially contains anti-NDAA antibodies, such as the AD-associated β-amyloid peptide. Other applications of the methods of the invention for research uses will be readily apparent to those skilled in the art.

The methods and kits of the invention are useful in a variety of diagnostic applications, such as the detection of anti-NDAA antibodies in a patient. Presence in a subject of certain immunoglobulin class(es) and subclass(es) of antibodies directed to specific neurodegenerative disease-associated antigens can be correlated with disease risk, onset, progression, and outcome, especially in the case of anti-β-amyloid antibodies and Alzheimer's disease. In certain embodiments, the methods and kits of the invention can be used to correlate the presence of anti-NDAA antibodies with the potential development or the actual existence of neurodegenerative disease in a subject in a manner predictive of neurodegenerative disease advancement. In one embodiment, the invention provides a method for prognosticating and/or monitoring the onset of Aβ or aiding in diagnosing Aβ in a subject, comprising the steps of exposing a biological fluid from the subject (whose biological fluid is believed to comprise an antibody that recognizes an AD-associated antigen) with the immobilized antigen associated with AD, exposing the biological fluid to a binding agent comprising a detectable moiety and capable of binding to at least one antibody, and detecting the detectable moiety for the purpose of correlating presence of the detected antibody from the biological fluid with the onset or presence of Aβ in the subject. In certain embodiments, diagnostic applications feature detection of a first class or subclass of antibodies and further feature detection of another or a second class of antibodies.

Other applications of the methods of the invention for research uses will be readily apparent to those skilled in the art. The contents of any patents, patent applications, and references cited throughout this specification are hereby incorporated by reference in their entireties.

The data presented in the Examples below demonstrate the high precision of the method of the instant invention, particularly for identifying Ig classes and IgG subclasses of anti-Aβ₄₂ antibodies present in patients and control populations of Aβ₄₂ immunization studies.

### Exemplification

Throughout the examples, the following materials and methods were used unless otherwise stated.

### Materials and Methods

In general, the practice of the present invention employs, unless otherwise indicated, conventional techniques of chemistry, molecular biology, recombinant DNA technology, and immunology (especially, e.g., immunoglobulin technology). See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); Current Protocols in Molecular Biology, eds. Ausubel et al., John Wiley & Sons (1992).

Sera

Sera used for development of ELISAs and assay qualification were obtained from multiple sources. The IgA, IgM and IgG ELISAs utilized sera from immunized humans, as well as, Cynomoglus monkeys. Due to the lack of cross reactivity of the murine monoclonals with monkey antibodies only human sera were used to develop the subclass ELISAs. Subjects were immunized with varying concentrations of AN1792 adjuvanted with STIMULON™ QS-21 (Antigenics, Framingham, Mass.). Serum converted plasma packs from the Red Cross were used as a source of non-immunized “negative” adult human sera. Pools of toddler sera from closed clinical studies were examined for naturally occurring antibodies to AN1792. Human intravenous immunoglobulin (IVIG) pools were obtained from multiple vendors, Sandoglobulin (Sandos), Panglobulin (NDC) and Gamimune N (Bayer). A chimeric monoclonal antibody, c3D6 (murine variable region and human constant region), specific to the first five amino acids of the AN1792 peptide was provided by Wyeth (Johnson-Wood, K. 1997).

Antigens

AN1792, the synthetic 1-42 amino acid form of the naturally occurring Aβ₄₂ protein (Glenner, 1984, Joachim, 1989), was manufactured by the American Peptide Company. Material was then reconstituted in 10 mM sodium glycinate (pH 9.0) and then characterized via HPLC. β-amyloid (40-1) reversimer was manufactured at California Peptide Research and reconstituted at Wyeth Pharmaceuticals. Human albumin Fraction V was obtained from Sigma. A nontoxic variant of diphtheria toxin, cross-reacting material 197 (“CRM₁₉₇”), as well as CRM₁₉₇ conjugated to the first 7 amino acids of AN1792 (Aβ₄₂1-7/CRM₁₉₇), and a 13 amino acid peptide from the variable region 2 (“VR2”) region of PorA protein (“VR2 porin peptide”) of Neisseria meningitidis serogroup B were obtained from Wyeth Pharmaceuticals. The first seven amino acids of β-amyloid were selected based on previous epitope mapping studies demonstrating the significance as a B cell epitope of the N-terminal region of AN1792 (Bard F. et al., 2003 Proc. Natl Acad. Sci. USA 100(4): 2023-28).

IgG, IgM and IgA ELISA Procedure

Optimal conditions with regard to antigen coating concentration were determined empirically using a chimeric monoclonal antibody c3D6 and a pool of immunized monkey sera with each different lot of antigen. The optimal coating concentration irrespective of antigen lot or batch was determined as 5 μg/ml. Optimal coating buffer was determined to be carbonate/bicarbonate buffer pH 9.6 with 0.02% sodium azide (sodium carbonate and bicarbonate, J. T. Baker; sodium azide, Sigma). Flat bottomed 96-well medium binding microtiter plates (Costar) were coated for 90 min at 37° C., followed by overnight (16 hours) incubation at 4° C. with 100 μl/well of AN1792. Plates were stored at 4° C. without blocking and used within 30 days of preparation or with blocking and used within 7 days. Antigen coated plates were washed 5 times (EL 405 microplate washer, Biotek Instruments Inc.) with 0.01M Tris buffered saline (TBS) containing 0.1% Brij-35 (Sigma) and blocked with PBS containing 1% skim milk (Difco/Becton Dickinson) and 0.3% Tween 20 (Sigma), pH 7.2 for 1 hour at room temperature. Serum standards and sera for testing were prepared at the appropriate dilutions in PBS containing 1% skim milk and 0.3% tween 20, and serially diluted (two or three fold) in the blocked plates, which were washed five times with 0.01M TBS containing 0.1% Brij-35. The starting dilution of test serum was 1/50. Three positive control sera and one negative control serum were used to assure the assay reproducibility.

Test, standard and control sera were incubated 2 hours at room temperature (16 hours 4° C. for IgM and IgA assay), and the plates were then washed five times with 0.01M TBS containing 0.1% Brij-35.1 Bound anti-AN1792 antibodies were detected using alkaline phosphatase conjugated goat anti-human IgG (BioSource AH10305), IgM FC_{5μ}, fragment specific (Jackson 109-055-129), or IgA α-chain specific (Jackson 109-055-011) antibodies diluted in PBS containing 1% skim milk and 0.3% Tween 20 (100 μl/well) for 2 hours at room temperature. Plates were washed 5 times with 0.01M TBS containing 0.1% Brij-35, and bound conjugate detected colorimetrically by using p-nitrophenyl phosphate (Sigma) in 1M diethanolamine, pH 9.6 (Sigma) at 1 mg/ml (100 μl/well). Color development was stopped after one hour (2 hours for IgA assay) by addition of 50 μl 3M NaOH (J. T. Baker) to all wells of the test plates. OD values were read with a SpectraMax Plus microtiter plate reader (Molecular Devices) at a wavelength of 405 nm with a 690 nm reference filter. Data were analyzed using linear regression comparing the log of the OD to the log of the sample dilution. Unknown sample titers were normalized to an ELISA standard at an OD of 0.3.

Competitive Inhibition ELISA

To ensure ELISA specificity, competitive inhibition assays were developed. The competitive inhibition assays are extensions of the standard ELISA procedures with the following modifications. Individual serum dilutions were selected from the upper half of the linear range of the dilution curve, targeting ODs around 1.0. Diluted sera were mixed with competitors at various concentrations (20 μg highest concentration) and then incubated 1 hour at room temperature (or 30 minutes at 37° C.), in presence or absence of the competitor. The antibody/competitor mixtures and control wells consisting of sera without the competitor were subsequently transferred to the antigen-coated microtiter plates and assays completed following the ELISA procedure detailed above. The competition rate was determined by comparison of the absorbencies of the sera in presence or absence of the competitor. Results were expressed as % inhibition=((A_{405 nm} serum alone—A_{405 nm} serum mixture with competitor)/A_{405 nm} serum alone)×100. Competitors included AN1792 peptide, AN1792 reversimer, Aβ₄₂1-7/CRM₁₉₇ conjugate, CRM₁₉₇ protein, VR2 porin peptide and Streptococcus pneumoniae polysaccharide (“Pneumo PS”). High levels of inhibition from the homologous antigen AN1792 (≧80%), and low levels of inhibition (≦20%) from heterologous and non-related antigens are indicative of high assay specificity.

IgG Subclass ELISA Procedure

Coating and assay buffers for the subclass ELISAs are similar to the IgA, IgG and IgM ELISAs previously described (see above). Starting dilution for the test sera was 1/50 with a 2-fold serial dilution of each duplicate sample. The assay controls and standards consist of human sera from AN1792 immunized adults. The majority of the standards and control are unique to their respective ELISA. Primary sera incubations were shown to be optimal at 4° C. for 20±2 hours. Upon completion of the primary incubation, plates were washed five times, and 100 μl of murine anti-human IgG₁₋₄ monoclonal antibody were added to each well. All monoclonals are commercially available; clone numbers include NL16 (HP6012), HP6014, HP6050 and HP6025 for Ig₁, IgG₂, IgG₃ and IgG₄ identification, respectively. The reagents used in the ELISA described were obtained from Skybio Limited (Cat. No. SPM-15015, IgG₁; Cat. No. M73013, IgG₂), ICN Pharmaceuticals (Cat. No. 630821, IgG₃) and Zymed Laboratories (Cat. No. 05-3801, IgG₄). Dilutions of each clone were empirically determined and varied from ELISA to ELISA. Following a 2 hour room temperature incubation, plates were washed and the murine monoclonals were detected using rabbit anti-mouse IgG conjugated to alkaline phosphatase (Zymed Laboratories, Cat. No. 61-0122). Again, working dilution of the conjugate was optimized for each subclass ELISA. Plates were incubated for 2 hours at room temperature and then washed. The substrate, p-nitrophenyl phosphate, was used to visualize the reaction. After 2 hours, 3N NaOH was used to stop the reaction and OD values were read at a wavelength of 405 nm with a reference of 690 nm. Antibody assignments for each subclass ELISA were normalized to an assay standard at an OD of 0.3. Due to limited human sera and the nature of the immune response to the AN1792 peptide a universal standard could not be formulated for direct subclass ELISA comparisons.

### EXAMPLE 1

### Precision Measurement of anti-AN1792 Antibodies in Test Subjects

Both within-day and between-day measurement precision was defined for each ELISA, in order to assure reproducibility of antibody assignments via the ELISA-based methods of the invention. Within-day (WD) precision values reflect the variability of measurement observed for different dilutions of the same sample(s) run in all positions of a plate(s), on the same day using the same equipment and the same standard curve. With one exception, the observed coefficients of variation (CVs) for such measurements were less than 10% (refer to Table I). Between-day (BD) precision values express the measurement variability observed for sera samples examined on different days, using different equipment, technicians and standard curves. CVs for between-day measurements were greater than for comparable within-day measurements; however, all observed CVs were less than 30%.

TABLE I
Measurement precision results for IgG, IgA and IgM ELISAs

| | Titer | S.D. | % CV |
|---|---|---|---|
| IgG precision (WD) | | | |
| C3D6 (mAb) | 5,166,676 | 421,379 | 8.16 |
| High control (monkey pool) | 262,838 | 20,986 | 7.98 |
| Medium control (monkey pool) | 49,146 | 2,331 | 4.74 |
| Low control (hybrid) | 2,064 | 177 | 8.58 |
| IgG precision (BD) | | | |
| C3D6 (mAb) | 5,377,379 | 1,292,160 | 24.0 |
| High control (monkey pool) | 211,362 | 44,525 | 21.1 |
| Medium control (monkey pool) | 41,034 | 11,611 | 28.3 |
| Low control (hybrid) | 1,761 | 274 | 15.6 |
| IgM precision (WD) | | | |
| Standard monkey pool 1 | 2,463 | 61.5 | 2.5 |
| Control (monkey pool) | 4,752 | 235 | 4.94 |
| Control (human) | 389 | 32.6 | 8.38 |
| IgM precision (BD) | | | |
| Standard monkey pool 1 | 2,591 | 212 | 8.16 |
| Control (monkey pool) | 4,892 | 377 | 7.71 |
| Control (human) | 431 | 60.3 | 13.98 |
| IgA precision (WD) | | | |
| Standard (monkey) | 173 | 9.2 | 5.33 |
| Control 1 (monkey) | 258 | 8.2 | 3.2 |
| Control 2 (monkey) | 81 | 13.78 | 17.1 |
| IgA precision (BD) | | | |
| Standard (monkey) | 177 | 10.2 | 5.8 |
| Control 1 (monkey) | 234 | 25.1 | 10.7 |
| Control 2 (monkey) | 95 | 21 | 22.6 |

WD=within day; BD=between day. Standard curves were highly reproducible for the IgG, IgM and IgA ELISAs. The reproducibility was consistent over a wide range of titers for controls made using both human and monkey sera. The low sample population for the IgA and IgM ELISA were a result of limited control volume.

### EXAMPLE 2

### Linearity/Parallelism of Measurements

To determine whether non-biased antibody assignments could be made by the methods of the present invention, the ELISA measurements of the standard, control and test sera groups were assessed for both linearity and parallelism across a range of quantitative values. Linearity was assessed by examining the correlation coefficient (r) between two variables—measured optical density values (ODs) and dilution. The relationship between OD and dilution was closest to linear (r→±1.0) for measured OD₄₀₅ values between 0.03 and 2.0, with at least three points used to define the line for an antibody assignment. In determining antibody assignments, any instances of samples exhibiting OD-dilution correlation coefficients of r<0.975 or r>−0.975 were remedied via reexamination by ELISA. All of the data shown in Table II thus have correlation coefficients of r≧0.975 or r≦−0.975.

Parallelism was assessed by testing for equality of slopes of sera (i.e., samples, controls and the standard) plotted using log/log regression analysis. Table II summarizes the slope data. Examining mean values for each individual ELISA reveals minimal differences between sample slopes, indicative of a high level of parallelism within each ELISA.

TABLE II
Parallelism of the IgG, IgA and IgM ELISAs

| | Mean | | | |
|---|---|---|---|---|
| | slope | Range | S.D. | % CV |
| IgG assay | | | | |
| Standard | −1.0208 | −0.9207 to −1.099 | 0.0303 | 2.97 |
| C3D6 | −0.9327 | −0.8712 to −1.0577 | 0.0271 | 2.90 |
| High control | −1.0360 | −0.9270 to −1.1604 | 0.0434 | 4.19 |
| Medium control | −1.0669 | −0.880 to −1.205 | 0.0419 | 3.92 |
| Low control | −1.0066 | −0.7154 to −1.1769 | 0.0422 | 4.19 |
| IgM assay | | | | |
| Standard | −0.9278 | −0.813 to −1.051 | 0.066 | 7.1 |
| Control 1 | −0.9122 | −0.812 to −1.024 | 0.055 | 6.06 |
| Control 2 | −1.0138 | −0.869 to −1.247 | 0.101 | 10.0 |
| IgA assay | | | | |
| Standard | −0.9319 | −0.802 to −1.111 | 0.055 | 5.95 |
| Control 1 | −0.8155 | −0.654 to −0.948 | 0.063 | 7.73 |
| Control 2 | −0.7950 | −0.618 to −1.045 | 0.090 | 11.28 |

### EXAMPLE 3

### Specificity of Measurements

Specificity of the AN1792 ELISA was examined by preincubating AN1792 immunized human sera (Table III) and AN1792 immunized monkey sera (Table IV) with either homologous antigen (AN1792) peptide or heterologous antigens such as the Aβ₄₂1-7/CRM₁₉₇ conjugate, reversimer AN1792 (40-1), CRM₁₉₇ protein, VR2 porin peptide and Pneumo PS, or human serum albumin. Competition with the Aβ₄₂1-7/CRM₁₉₇ conjugate was similar to that observed for full length AN1792. While the kinetics of the reaction showed that less of the full length peptide was needed to compete with the antigen bound on the plate, both competitors eventually result in 100% competition as competitor concentration is increased, indicating that the bulk of the human immune response to the 42 amino acid peptide is to the first seven amino acids of the peptide. High levels of competitive inhibition from the homologous antigen and low levels of competitive inhibition from the unrelated antigens were indicative of superior assay specificity.

TABLE III
IgG, IgM and IgA competition ELISA using immunotherapy human sera

| | | competitor | | | | |
|---|---|---|---|---|---|---|
| | | % inhibition | | | | |
| Compet. | competitor | | Aβ₄₂(1-7)/ | AN1792 | | |
| range | concentration | AN1792 | CRM₁₉₇ | reversimer | | VR2 porin |
| (%) | (ug/ml) | (1-42) | conjugate | (42-1) | CRM₁₉₇ | peptide |
| IgG | 10 | 94.0 ± 1.51 | ND | −0.3 ± 4.41 | −0.3 ± 4.41 | 1.1 ± 2.96 |
| assay | | | | | | |
| IgM | 10 | 100.23 ± 0.31 | 100.08 ± 0.21 | −4.80 ± 5.13 | 1.75 ± 5.37 | 6.97 ± 8.50 |
| assay | 1 | 99.24 ± 1.83 | 99.05 ± 0.63 | 0.56 ± 7.98 | ND | ND |
| | 0.1 | 99.81 ± 0.43 | 96.12 ± 0.87 | 9.18 ± 8.33 | ND | ND |
| | 0.01 | 82.02 ± 5.64 | 69.17 ± 9.28 | 11.62 ± 2.82 | ND | ND |
| IgA | 10 | 102.06 ± 3.02 | 99.5 ± 4.71 | −8.62 ± 4.75 | −1.49 ± 0.82 | 6.39 ± 3.71 |
| assay | 1 | 100.75 ± 3.38 | 90.18 ± 10.02 | 3.57 ± 7.76 | ND | ND |
| | 0.1 | 93.76 ± 5.02 | 81.25 ± 6.33 | 6.39 ± 11.04 | ND | ND |
| | 0.01 | 45.12 ± 10.45 | 45.78 ± 22.03 | 9.58 ± 6.96 | ND | ND |

TABLE IV
IgG, IgM and IgA competition ELISA
using immunotherapy monkey sera

| | | competitor | | | |
|---|---|---|---|---|---|
| | | % inhibition | | | |
| Compet. | competitor | | AN1792 | | |
| range | concentration | AN1792 | reversimer | | Pneumo |
| (%) | (ug/ml) | (1-42) | (42-1) | CRM₁₉₇ | PS |
| IgG | 10 | 96.75 | ND | −0.55 | 2.73 |
| assay | 1 | 93.15 | ND | −1.35 | −0.43 |
| | 0.1 | 79.05 | ND | −0.13 | 0.35 |
| | 0.01 | 38.93 | ND | −1.85 | 0.38 |
| IgM | 10 | 100.37 | −1.63 | 0.79 | 1.98 |
| assay | 1 | 100.02 | −5.09 | −5.14 | ND |
| | 0.1 | 96.81 | 0.79 | −0.94 | ND |
| | 0.01 | 36.42 | 7.67 | 4.93 | ND |
| IgA | 10 | 101.06 | 3.22 | 1.35 | 7.78 |
| assay | 1 | 90.95 | 4.85 | −0.93 | ND |
| | 0.1 | −9.29 | 11.43 | 6.15 | ND |
| | 0.01 | −11.9 | 13.69 | 9.16 | ND |

Non-immunized human adult sera, as well as toddler sera from closed clinical 5 studies were screened using the IgG, IgA and IgM ELISAs to examine the frequency of preexisting antibodies to AN1792 (Table V). Only IgM antibodies were detected in the samples tested. Approximately 40% of human adults had preexisting IgM antibodies, whereas a much lower number of toddlers had raised antibodies to β-amyloid. The table indicates <5% of the toddler sera tested positive for anti-β-amyloid IgM; however, the 10 actual percentage is much lower since pooled sera were examined. The age of the toddler sera ranged from 2 to 30 months; the one positive sample was from the 24 month pool. Since the adult sera were acquired from the Red Cross, information pertaining to each serum sample was unavailable.

TABLE V
AN1792 antibodies in non-immunotherapy human sera

| | | | Total number of |
|---|---|---|---|
| ELISA | Top OD range* | % positive | screened sera |
| | Adult sera | | |
| IgG | 0.018-0.106 | 0 | 23 |
| IgM | 0.018-1.935 | 40 | 42 |
| IgA | 0.007-0.027 | 0 | 21 |
| | Toddler sera | | |
| IgM | 0-0.32 | 2 | 51** |

*Following subtraction of blanks
**51 pools were screened. Approximately 10 sera were used to make 1 pool.

### EXAMPLE 4

### Sensitivity of Measurements

Sensitivity, as gauged by plate lower limits, was determined for all ELISAs. These theoretical lower limits were not used when reporting results. Instead the more conservative value, half of the starting dilution, was used to categorize samples as positive or negative. The IgG ELISA used a 1:100 starting dilution for unknown samples; unknowns that failed to give a top OD of 0.3 were assigned a titer of <100. All other ELISAs used a 1:50 starting dilution for unknowns; a titer of <50 was used to represent a negative sample.

### EXAMPLE 5

### IgG Subclass ELISA Development and Qualification

The ELISA assays of the present invention are capable of distinguishing IgG subclasses of human antibodies with great specificity. Similar assessments were performed when developing the subclass ELISAs as for the isotyping ELISAs. Coating conditions, assay kinetics, and dilutions of reagents were examined and optimized. Additionally, since the subclass ELISAs used a tertiary reporting system, specificity of the monoclonals used to detect human subclasses were confirmed by coating microtiter plate wells with purified human subclass proteins (IgG_{1, 2, 3 or 4}), followed by the addition of the subclassing monoclonals. Results showed no to minimal (<1.0%) cross reactivity between each of the monoclonal antibodies, demonstrating the ability of the invention to correctly identify the IgG subclass of interest (Data not shown).

Precision, linearity, parallelism and specificity were all examined for IgG subclass ELISAs. Limited control and standard volumes interfered with precision of some measurements. Within day precision studies gave higher CVs than between day readings for many samples. Additionally, only one competitor concentration was examined with one sera dilution for each sample. Table VI summarizes the results obtained during qualification of the subclass ELISA.

TABLE VI
Subclass ELISA qualification summary

| Qualification | | |
|---|---|---|
| parameter | Sample(s) | Results |
| WD Precision | Subclass standards | % CVs ranged from a |
| | Subclass controls | low of 3.3% to a high |
| | | of 24%. |
| BD Precision | All subclass standards | % CVs were all less |
| | All subclass controls | than 20%. |
| Specificity | 21 sera from clinical study | Homologous |
| | A pool of 16 sera from | competition using |
| | clinical study | AN1792 ranged from a |
| | | low of 60% to a high |
| | | of 96.6%. Competition |
| | | using unrelated |
| | | antigens was minimal. |
| Limit of | | 1:50 (Assigned LOQ). |
| Quantitation (LOQ) | | |
| (Sensitivity) | | |
| Linearity/parallelism | All subclass standards | The ELISA was found |
| | All subclass controls | to be both linear, as |
| | | well as, demonstrate |
| | | parallelism. |

WD = within day;
BD = between day.

Fifty-eight (58) clinical samples previously determined to be positive (OD₄₀₅>0.3 when tested at 1:100) for anti-AN1792 total IgG were analyzed using the four subclass ELISAs. The sera samples originated from adults with mild to moderate Alzheimer's disease. The samples chosen were a non-randomized subset selected for laboratory assay development and thus not necessarily representing clinical population features. Individuals received one to three immunizations with an AN1792/QS-21 formulation. Table VII summarizes the results from the analysis. All of the individuals immunized generated IgG₁ antibodies to AN1792 (Aβ) antigen. 37/58 (63.8%) had IgG₂ anti-AN1792 antibodies, 24/58 (41.4%) had IgG₃ anti-AN1792 antibodies, and only 1/58 (1.7%) had anti-AN1792 IgG₄ antibodies.

TABLE VII
Subclass data: IgG subclass distributions in a subset of samples
previously shown to be positive for total IgG and IgM

| IgG Subclass | Number positive | Percentage positive (%) |
|---|---|---|
| IgG₁ | 58/58 | 100 |
| IgG₂ | 37/58 | 63.8 |
| IgG₃ | 24/58 | 41.4 |
| IgG₄ | 1/58 | 1.7 |

### Equivalents

For one skilled in the art, using no more than routine experimentation, there are many equivalents to the specific embodiments of the invention described herein. Such equivalents are intended to be encompassed by the following claims.

We claim:

1. A method for determining the level of an anti-Aβ antibody of a particular immunoglobulin (Ig) class or subclass produced in a patient immunized with an Aβ immunogenic composition, comprising:
contacting a biological sample from the patient with an immobilized Aβ antigen under conditions sufficient for binding of the anti-Aβ antibody to the antigen in the sample, if present, followed by washing to remove unbound biological sample;
contacting the bound anti-Aβ antibody, if present, with an agent capable of specifically binding to the antibody, followed by washing to remove unbound agent;
quantifying the bound agent, wherein the quantity of agent bound indicates an amount of anti-Aβ antibody present in the biological sample,
such that the level of the anti-Aβ antibody of a particular Ig class or subclass is determined.

2. The method of claim 1, wherein the Aβ antigen is Aβ1-42.

3. The method of claim 1, wherein the Aβ antigen is AN1792.

4. The method of claim 1, wherein the level of the anti-Aβ antibody of a particular Ig class is determined.

5. The method of claim 4, wherein the agent capable of specifically binding to the antibody is a second antibody which is detectable.

6. The method of claim 5, wherein the second antibody is enzyme-linked.

7. The method of claim 6, wherein quantifying the enzyme-linked second antibody comprises detecting an activity of said enzyme-linked antibody, wherein a level of said activity indicates a level of said bound anti-Aβ antibodies.

8. The method of claim 1, wherein the level of the anti-Aβ antibody of a particular Ig subclass is determined.

9. The method of claim 8, wherein the agent capable of specifically binding to the antibody is a second antibody.

10. The method of claim 9, wherein quantifying the second antibody comprises exposing the second antibody to a third antibody which is detectable.

11. The method of claim 10, wherein the third antibody is enzyme-linked.

12. The method of claim 11, wherein quantifying the enzyme-linked third antibody comprises detecting an activity of said antibody, wherein a level of said activity indicates a level of said bound anti-Aβ antibodies.

13. The method of claim 4, wherein the anti-Aβ antibody is of the IgG, IgA, IgM or IgE class.

14. The method of claim 8, wherein the anti-Aβ antibody is of the IgG₁, IgG₂, IgG₃ or IgG₄ subclass.

15. A method for determining the efficacy of a β-amyloid immunotherapy in a patient, comprising:
assaying for class-specific or subclass specific anti-β-amyloid antibodies in a biological sample from said patient;
wherein a level of said isotype-specific or subclass-specific antibodies is determinative of the efficacy of said β-amyloid immunotherapy.

16. The method of claim 15, wherein assaying for isotype-specific or subclass specific anti-β-amyloid antibodies comprises:
contacting said biological sample with immobilized β-amyloid under conditions sufficient for binding of said β-amyloid to said class-specific or subclass specific anti-β-amyloid antibodies, if present, followed by washing to remove unbound biological sample;
contacting the bound anti-β-amyloid antibodies, if present, with an enzyme-linked agent which specifically binds to the antibodies, followed by washing to remove unbound agent; and
quantifying the enzyme-linked agent, wherein the quantity of said enzyme-linked agent indicates a level of said bound anti-β-amyloid antibodies,
such that isotype-specific or subclass specific anti-β-amyloid antibodies are assayed.

17. The method of claim 16, wherein the immobilized β-amyloid is Aβ₁₋₄₂.

18. The method of claim 17, wherein the immobilized β-amyloid is AN1792.

19. The method of claim 15, wherein the β-amyloid immunotherapy comprises administering a β-amyloid immunogenic composition to said patient.

20. The method of claim 19, wherein the β-amyloid immunogenic composition comprises Aβ₁₋₄₂.

21. The method of claim 19, wherein the β-amyloid immunogenic composition comprises AN1792.

22. The method of claim 20 or 21, wherein the immunogenic composition further comprises an adjuvant.

23. The method of claim 22, wherein the adjuvant is STIMULON™ QS-21.

24. The method of claim 15 or 16, wherein class-specific antibodies are assayed in said biological sample.

25. The method of claim 24, wherein IgG, IgA or IgM antibodies are assayed in said biological sample.

26. The method of claim 25, wherein an increase in the level of IgG, IgA or IgM antibodies following initiation of said immunotherapy is determinative of the efficacy of said β-amyloid immunotherapy.

27. The method of claim 15 or 16, wherein said level is compared to the level of said antibodies present in a suitable control.

28. The method of claim 15 or 16, wherein subclass-specific antibodies are assayed in said biological sample.

29. The method of claim 28, wherein IgG₁, IgG₂, IgG₃ or IgG₄ antibodies are assayed.

30. The method of claim 28, wherein an increase in the level of IgG₁, IgG₂, IgG₃ or IgG₄ antibodies following initiation of said immunotherapy is determinative of the efficacy of said β-amyloid immunotherapy.

31. The method of claim 15 or 16, wherein the biological sample is a serum sample.

32. The method of claim 15 or 16, wherein the biological sample is a cerebrospinal fluid (CSF) sample.

33. The method of claim 15 or 16, wherein the subject has or is at risk for an amyloidogenic disease.

34. The method of claim 15 or 16, wherein the subject has or is at risk for Alzheimer's Disease.

35. A kit comprising an immobilized Aβ antigen or Aβ antigen suitable for immobilization, an agent capable of specifically binding to an anti-Aβ antibody, and directions for use.

36. The kit of claim 35, wherein the Aβ antigen is Aβ1-42.

37. The kit of claim 35, wherein the Aβ antigen is AN1792.

38. The kit of claim 35, wherein the agent is capable of specifically binding to an anti-Aβ antibody of a particular Ig class.

39. The kit of claim 38, wherein the agent capable of specifically binding to the anti-Aβ antibody is a second antibody which is detectable.

40. The kit of claim 39, wherein the second antibody is enzyme-linked.

41. The kit of claim 40, further comprising agents suitable for detecting activity of the enzyme.

42. The kit of claim 35, wherein the agent is capable of specifically binding to an anti-Aβ antibody of a particular Ig subclass.

43. The kit of claim 42, wherein the agent capable of specifically binding to the anti-Aβ antibody is a second antibody.

44. The kit of claim 43, further comprising a third antibody capable of binding the second antibody.

45. The kit of claim 44, wherein the third antibody is enzyme-linked.

46. The kit of claim 45, further comprising agents suitable for detecting activity of the enzyme.

47. The kit of claim 38, wherein the anti-Aβ antibody is of the IgG, IgA, IgM or IgE class.

48. The kit of claim 42, wherein the anti-Aβ antibody is of the IgG₁, IgG₂, IgG₃, IgG4 or subclass.
